(19) United States
(12) Patent Application Publication
(10) Pub. No.: US 2006/0073488 A1
(43) Pub. Date: Apr. 6, 2006

(54) Method and standards for detecting binding to an array

(76) Inventors: Nicholas M. Sampas, San Jose, CA (US); Robert A. Ach, San Francisco, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/959,258

(22) Filed: Oct. 5, 2004

(51) Int. Cl. C12Q 1/68

(52) U.S. Cl. 435/6

(57) ABSTRACT

The present invention relates to methods for detecting, quantitating, or standardizing binding to a nucleic acid array, standard polynucleotides that can be used in such methods, compositions including the standard polynucleotides, and methods of making the standard polynucleotides. An aspect of the present invention relates to a complete exon transcript of a gene. The gene can be a gene expressed as multiple transcripts but encoding only transcripts lacking at least a portion of at least one exon. A complete exon transcript includes all of the known bases of every known exon for such a gene. An aspect of the present invention relates to a method of detecting binding to a nucleic acid array. The method can include contacting the nucleic acid array with a standard polynucleotide for a gene. The standard polynucleotide for a gene includes all of the known bases of every known exon for that gene. In an embodiment, the standard polynucleotide includes a complete exon transcript of the gene.

### BACKGROUND OF THE INVENTION

Conventional methods and standards for detecting binding to an array can be difficult or impossible to standardize or quantify. For example, one commonly used reference sample for a nucleic acid array is expressed RNA from a particular cell culture. Although the RNA can be expressed in large quantities, the relative amounts of the various RNAs in the sample cannot be selected. Unfortunately, each batch of RNA expressed can be quite a bit different from each other batch. Therefore, standardization and quantification can be difficult or impossible. Further, certain genes in the cell culture will not be expressed and, thus, cannot be detected upon hybridization.

There remains a need for improved methods for detecting or quantifying binding to or standardizing nucleic acid arrays.

### SUMMARY OF THE INVENTION

The present invention relates to methods for detecting, quantitating, or standardizing binding to a nucleic acid array, standard polynucleotides that can be used in such methods, compositions including the standard polynucleotides, and methods of making the standard polynucleotides.

An aspect of the present invention relates to a complete exon transcript of a gene. The gene can be a gene expressed as multiple transcripts but encoding only transcripts lacking at least a portion of at least one exon. A complete exon transcript includes all of the known bases of every known exon for such a gene.

An aspect of the present invention relates to a method of detecting binding to a nucleic acid array. The method can include contacting the nucleic acid array with a standard polynucleotide for a gene. The standard polynucleotide for a gene can include all of the known bases of every known exon for that gene. In an embodiment, the standard polynucleotide includes a complete exon transcript of the gene. In an embodiment, the standard polynucleotide includes a differential exon transcript of the gene. A differential exon transcript includes contiguous nucleotides of a complete exon transcript but less than the entire complete exon transcript, the contiguous nucleotides being found in a first transcript but not in a second transcript. The method can also include monitoring for detectable binding of the standard polynucleotide to the array. The method can include providing a nucleic acid array including a plurality of features at locations on a substrate. The method can include forwarding to a remote location a result obtained by the method. The method can include transmitting data representing a result of a reading obtained by the method.

An aspect of the present invention relates to a composition including a standard polynucleotide. In an embodiment, the composition includes complete exon transcript of a gene. In an embodiment, the composition includes a differential exon transcript of a gene. The composition can also include other materials, such as an additional polynucleotide, a nucleic acid array, or a buffer composition.

An aspect of the present invention relates to a method of making a complete exon transcript for a gene. This method includes determining the sequence of nucleotides making up a complete exon transcript for a gene. The method can include producing a polynucleotide including the sequence of nucleotides of the complete exon transcript. The method can include providing the sequence of the complete exon transcript in a format readable by a machine or by a human. In an embodiment, the present invention relates to a method of making a differential exon transcript for a gene. This embodiment of the method includes determining the sequence of nucleotides making up a differential exon transcript for a gene. This embodiment can include producing a polynucleotide including the sequence of nucleotides of the differential exon transcript. This embodiment can include providing the sequence of the differential exon transcript in a format readable by a machine or by a human.

An aspect of the present invention relates to a computer program product for use with a method and apparatus such as described herein. The program product includes a computer readable storage medium having a computer program stored thereon and which, when loaded into a programmable processor, provides instructions to the processor of that apparatus such that it will execute the procedures required of it to perform a method of the present invention.

### BRIEF DESCRIPTION OF THE FIGURES

FIG. 1 schematically illustrates a gene encoding two splice variant mRNAs, labeled 1 and 2. This figure also schematically illustrates a standard polynucleotide (SP) for this gene. In this case, mRNA 1has the same sequence as the standard polynucleotide.

FIG. 2 schematically illustrates a standard polynucleotide (SP) that is a complete exon transcript for a gene encoding three splice variant mRNAs.

FIG. 3 schematically illustrates a standard polynucleotide (SP) that is a complete exon transcript for a gene encoding 20 splice variant mRNAs.

### DETAILED DESCRIPTION

### DEFINITIONS

In the present application, unless a contrary intention appears, the following terms refer to the indicated characteristics.

As used herein, the term “exon” refers to any coding or messenger sequence of deoxynucleotides, such as any intragenic region of DNA in eukaryotes that will be ultimately expressed in mRNA or rRNA residues.

As used herein, the term “nucleic acid” refers to a polymer made up of nucleotides, e.g., deoxyribonucleotides or ribonucleotides.

As used herein, the terms “ribonucleic acid” and “RNA” refers to a polymer that includes at least one ribonucleotide at its 3′ end, e.g., a polymer made up completely of ribonucleotides.

As used herein, the terms “deoxyribonucleic acid” and “DNA” refers to a polymer made up of deoxyribonucleotides.

As used herein, the term “polynucleotide” includes a nucleotide multimer having any number of nucleotides (for example 10 to 200, or more). This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. For example, a polynucleotide can include DNA (including cDNA), RNA, oligonucleotides, and PNA and other polynucleotides as described in U.S. Pat. No. 5,948,902 and references cited therein (all of which are incorporated herein by reference), regardless of the source.

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides.

An “oligonucleotide” generally refers to a nucleotide multimer of about 10 to 100 nucleotides in length.

As used herein, the phrases “percent (%) nucleic acid sequence identity” and “% nucleic acid sequence identity” used with respect to the complete exon transcript, the differential exon transcript, the standard polynucleotide, or the complement thereof refers to the percentage of nucleotides in a sequence of interest that are identical with the nucleotides in the complete exon transcript, the differential exon transcript, the standard polynucleotide, or the complement thereof after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms. Sequence comparison programs including the publicly available NCBI-BLAST2 (Altschul et al., Nucleic Acids Res. 25:3389-3402 (1997)) can be employed for calculating % identity with its search parameters set to default values.

As used herein, the phrase “stringent conditions” or “high stringency conditions” for hybridization of oligo or polynucleotides include, for example: washing at low ionic strength and high temperature, e.g., 0.015 M sodium chloride/0.0015 M sodium citrate/0.1% sodium dodecyl sulfate at 50° C.; hybridization in the presence of a denaturing agent, e.g., 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or hybridizing in 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 g/ml), 0.1% SDS, and 10% dextran sulfate at 42° C. and washing at 42° C. in 0.2×SSC (sodium chloride/sodium citrate) and 50% formamide followed by washing with 0.1×SSC containing EDTA at 55° C.

Hybridizing” and “binding”, with respect to polynucleotides, are used interchangeably. “Hybridizing conditions” for a polynucleotide array refer to suitable conditions of time, temperature and the like, such that a target sequence present in solution will bind to an array feature carrying a complementary sequence to a greater extent than to features carrying only sequences which are not complementary to the target sequence (and preferably at least 20% or 100%, or even 200 or 500% greater).

As used herein, the term “isolated” polynucleotide refers to a polynucleotide that is identified and separated from at least one component of its natural environment. A recombinantly produced or synthetic polynucleotide is an isolated polynucleotide.

As used herein, the phrase “control sequences” refers to DNA sequences controlling transcription of an operably linked polynucleotide in a selected host cell or organism.

As used herein, the phrase “operably linked” refers to a polynucleotide in a functional relationship with another polynucleotide.

An “array”, unless a contrary intention appears, includes any one-, two- or three-dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other). Target probes may be covalently bound to a surface of a non-porous or porous substrate either directly or through a linker molecule, or may be adsorbed to a surface using intermediate layers (such as polylysine) or porous substrates.

An “array layout” refers to one or more characteristics of the array or the features on it. Such characteristics include one or more of: feature positioning on the substrate; one or more feature dimensions; some indication of an identity or function (for example, chemical or biological) of a moiety at a given location; how the array should be handled (for example, conditions under which the array is exposed to a sample, or array reading specifications or controls following sample exposure).

A “pulse jet” is a device which can dispense drops in the formation of an array. Pulse jets operate by delivering a pulse of pressure to liquid adjacent an outlet or orifice such that a drop will be dispensed therefrom (for example, by a piezoelectric or thermoelectric element positioned in a same chamber as the orifice).

An array “package” may be the array plus only a substrate on which the array is deposited, although the package may include other features (such as a housing with a chamber).

A “chamber” references an enclosed volume (although a chamber may be accessible through one or more ports).

A “region” refers to any finite small area on the array that can be illuminated and any resulting fluorescence therefrom simultaneously (or shortly thereafter) detected, for example a pixel.

A “processor” references any hardware and/or software combination which will perform the functions required of it. For example, any processor herein may be a programmable digital microprocessor such as available in the form of a mainframe, server, or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic or optical disk may carry the programming, and can be read by a suitable disk reader communicating with each processor at its corresponding station.

It will also be appreciated that throughout the present application, that words such as “top”, “upper”, and “lower” are used in a relative sense only.

When one item is indicated as being “remote” from another, this is referenced that the two items are at least in different buildings, and may be at least one mile, ten miles, or at least one hundred miles apart. “Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

Reference to a singular item, includes the possibility that there are plural of the same items present.

“May” means optionally.

Methods recited herein may be carried out in any order of the recited events which is logically possible, as well as the recited order of events.

All patents and other references cited in this application, are incorporated into this application by reference except insofar as they may conflict with those of the present application (in which case the present application prevails).

Method of Detecting Binding to a Nucleic Acid Array

An embodiment includes a method for detecting binding to an array, specifically a nucleic acid array. The method includes contacting a nucleic acid array with a standard polynucleotide. The method can include detecting binding of the standard polynucleotide to the array. In an embodiment, the standard polynucleotide is selected to provide enhanced detection of a gene or gene transcript.

In an embodiment, the standard polynucleotide includes all of the nucleotides that are transcribed for a particular gene. For example, the standard polynucleotide can include all of the nucleotides that are transcribed for a particular gene under any circumstances. For example, the standard polynucleotide can include all of the bases of every exon for a gene. All of the exons transcribed into any RNA are in the standard polynucleotide. For example, the standard polynucleotide can include all of the known bases of every known exon for a gene. All of the known exons transcribed into any RNA are in the standard polynucleotide. The longest transcribed sequence of nucleotides for each exon is in the standard polynucleotide and the exons are in genomic order or reverse genomic order. In an embodiment, the standard polynucleotide does not include any intron sequences.

In certain embodiments, the standard polynucleotide can be diagnostic of a particular cell, cell type, a plurality of cell types, organism or individual, species, genus, order, or the like. For example, the standard polynucleotide can be diagnostic of a gene found in a cell, such as a particular isolate of a microorganism. Such a standard polynucleotide can include all of the nucleotides that are transcribed diagnostic of a particular gene in a cell, e.g., a particular isolate of a microorganism. For example, the standard polynucleotide can be diagnostic of a gene found in a cell, such as a CD4 T-cell. Such a standard polynucleotide can include all of the nucleotides that are transcribed diagnostic of a particular gene in a cell, e.g., a CD4 T-cell. For example, the standard polynucleotide can be diagnostic of a gene found in a type of cell, such as a Staphylococcus cell that can produce endotoxin. Such a standard polynucleotide can include all of the nucleotides that are transcribed diagnostic of a particular gene in a type of cell, e.g., a Staphylococcus cell that can produce endotoxin. For example, the standard polynucleotide can be diagnostic of a gene found in a type of cell, such as a T-cell. Such a standard polynucleotide can include all of the nucleotides that are transcribed diagnostic of a particular gene in a type of cell, e.g., a T-cell. For example, the standard polynucleotide can be diagnostic of a gene found in a plurality of cell types, such as a tissue or organism. Such a standard polynucleofide can include all of the nucleotides that are transcribed diagnostic of a particular gene in a plurality of cell types of cell, e.g., a tissue or organism. For example, the standard polynucleotide can be diagnostic of a gene found in a organism or individual, such as a particular laboratory animal or an individual human. Such a standard polynucleotide can include all of the nucleotides that are transcribed diagnostic of a particular gene in a particular organism or individual, e.g., a laboratory animal or an individual human. For example, the standard polynucleotide can be diagnostic of a gene found in a species, such as humans. Such a standard polynucleotide can include all of the nucleotides that are transcribed diagnostic of a particular gene, e.g., a human gene. For example, the standard polynucleotide can be diagnostic of a gene found in a genus, such as the genus pan (chimpanzee and bonobo). Such a standard polynucleotide can include all of the nucleotides that are transcribed diagnostic of a particular gene in a genus, e.g., the genus pan. For example, the standard polynucleotide can be diagnostic of a gene found in an order of organisms, such as primates. Such a standard polynucleotide can include all of the nucleotides that are transcribed diagnostic of a particular gene in an order, e.g., primates.

In certain embodiments, the standard polynucleotide can be diagnostic of a particular growth condition, environmental condition, developmental stage, or the like. For example, the standard polynucleotide can be diagnostic of a gene of interest in a particular growth condition, such as in a particular mammalian cell grown in the presence of an interleukin. Such a standard polynucleotide can include all of the nucleotides that are transcribed diagnostic of a particular gene in a particular growth condition, e.g., in a mammalian cell grown in the presence of an interleukin. For example, the standard polynucleotide can be diagnostic of a gene of interest in a particular environmental condition, such as a gene of interest in maize grown under drought conditions. Such a standard polynucleotide can include all of the nucleotides that are transcribed diagnostic of a particular gene in a particular environmental condition, e.g., in maize grown under drought conditions. For example, the standard polynucleotide can be diagnostic of a gene of interest in a particular developmental stage, such a gene of interest in an insect larvae. Such a standard polynucleotide can include all of the nucleotides that are transcribed diagnostic of a particular gene in a particular developmental stage, e.g., in an insect larvae.

In certain embodiments, the standard polynucleotide can be diagnostic of a particular subset of exons or splice variants. The subset of exons or splice variants can be those expressed in a particular cell, cell type, organism or individual, species, genus, order, or the like. The subset of exons or splice variants can be those expressed in a particular growth condition, environmental condition, developmental stage, or the like.

For a gene that is expressed as only a single transcript, the standard polynucleotide is the mRNA, cDNA, or a complement thereof.

A minority of genes expressed as multiple transcripts encode a transcript that includes all of the known bases of all of the known exons. For such a gene, the standard polynucleotide is the mRNA, cDNA, or complement thereof that includes all of the known bases of all of the known exons.

A majority of genes expressed as multiple transcripts encode only transcripts lacking at least a portion of at least one exon. In such a gene, the longest encoded transcript lacks at least a portion of at least one exon. For such genes, the present standard polynucleotide includes a sequence of nucleic acids not found in nature. As used herein, such a standard polynucleotide is referred to as a “complete exon transcript” of the gene. The complete exon transcript can be a polynucleotide such as RNA or DNA and can be a plus sense strand or a minus sense strand.

In certain embodiments, the complete exon transcript can be diagnostic of a particular cell, cell type, organism or individual, species, genus, order, or the like. For example, the complete exon transcript can be diagnostic of a gene found in a cell, such as a particular isolate of a microorganism. Such a complete exon transcript can include all of the nucleotides that are transcribed diagnostic of a particular gene in a cell, e.g., a particular isolate of a microorganism. For example, the complete exon transcript can be diagnostic of a gene found in a cell, such as a CD4 T-cell. Such a complete exon transcript can include all of the nucleotides that are transcribed diagnostic of a particular gene in a cell, e.g., a CD4 T-cell. For example, the complete exon transcript can be diagnostic of a gene found in a type of cell, such as a Staphylococcus cell that can produce endotoxin. Such a complete exon transcript can include all of the nucleotides that are transcribed diagnostic of a particular gene in a type of cell, e.g., a Staphylococcus cell that can produce endotoxin. For example, the complete exon transcript can be diagnostic of a gene found in a type of cell, such as a T-cell. Such a complete exon transcript can include all of the nucleotides that are transcribed diagnostic of a particular gene in a type of cell, e.g., a T-cell. For example, the complete exon transcript can be diagnostic of a gene found in a plurality of cell types, such as a tissue or organism. Such a complete exon transcript can include all of the nucleotides that are transcribed diagnostic of a particular gene in a plurality of cell types of cell, e.g., a tissue or organism. For example, the complete exon transcript can be diagnostic of a gene found in a organism or individual, such as a particular laboratory animal or an individual human. Such a complete exon transcript can include all of the nucleotides that are transcribed diagnostic of a particular gene in a particular organism or individual, e.g., a laboratory animal or an individual human. For example, the complete exon transcript can be diagnostic of a gene found in a species, such as humans. Such a complete exon transcript can include all of the nucleotides that are transcribed diagnostic of a particular gene, e.g., a human gene. For example, the complete exon transcript can be diagnostic of a gene found in a genus, such as the genus pan (chimpanzee and bonobo). Such a complete exon transcript can include all of the nucleotides that are transcribed diagnostic of a particular gene in a genus, e.g., the genus pan. For example, the complete exon transcript can be diagnostic of a gene found in an order of organisms, such as primates. Such a complete exon transcript can include all of the nucleotides that are transcribed diagnostic of a particular gene in an order, e.g., primates.

In certain embodiments, the complete exon transcript can be diagnostic of a particular growth condition, environmental condition, developmental stage, or the like. For example, the complete exon transcript can be diagnostic of a gene of interest in a particular growth condition, such as in a particular mammalian cell grown in the presence of an interleukin. Such a complete exon transcript can include all of the nucleotides that are transcribed diagnostic of a particular gene in a particular growth condition, e.g., in a mammalian cell grown in the presence of an interleukin. For example, the complete exon transcript can be diagnostic of a gene of interest in a particular environmental condition, such as a gene of interest in maize grown under drought conditions. Such a complete exon transcript can include all of the nucleotides that are transcribed diagnostic of a particular gene in a particular environmental condition, e.g., in maize grown under drought conditions. For example, the complete exon transcript can be diagnostic of a gene of interest in a particular developmental stage, such a gene of interest in an insect larvae. Such a complete exon transcript can include all of the nucleotides that are transcribed diagnostic of a particular gene in a particular developmental stage, e.g., in an insect larvae.

In certain embodiments, the complete exon transcript can be diagnostic of a particular subset of exons or splice variants, so long as the longest encoded transcript lacks at least a portion of at least one exon of the subset. The subset of exons or splice variants can be those expressed in a particular cell, cell type, organism or individual, species, genus, order, or the like. The subset of exons or splice variants can be those expressed in a particular growth condition, environmental condition, developmental stage, or the like.

A transcript lacking at least a portion of at least one exon can be encoded by, for example, a gene that encodes multiple splice variant transcripts, a gene with alternative polyadenylation sites, or a gene with multiple start sites. For example, a majority of genes expressed as multiple splice-variant transcripts encode only transcripts lacking at least a portion of at least one exon found in one or more of the other splice variant transcripts. The longest encoded splice variant transcript lacks at least a portion of at least one exon found in one or more of the other splice variant transcripts. For example, a majority of genes with alternative polyadenylation sites encode only transcripts lacking at least a portion of at least one exon found in one or more of the alternative transcripts. The longest encoded transcript lacks at least a portion of at least on exon found in one or more of transcript with a different polyadenylation site. For example, a majority of genes with multiple start sites encode only transcripts lacking at least a portion of at least one exon found in one or more of the several transcripts. The longest encoded transcript lacks at least a portion of at least on exon found in one or more of transcript with a different start site.

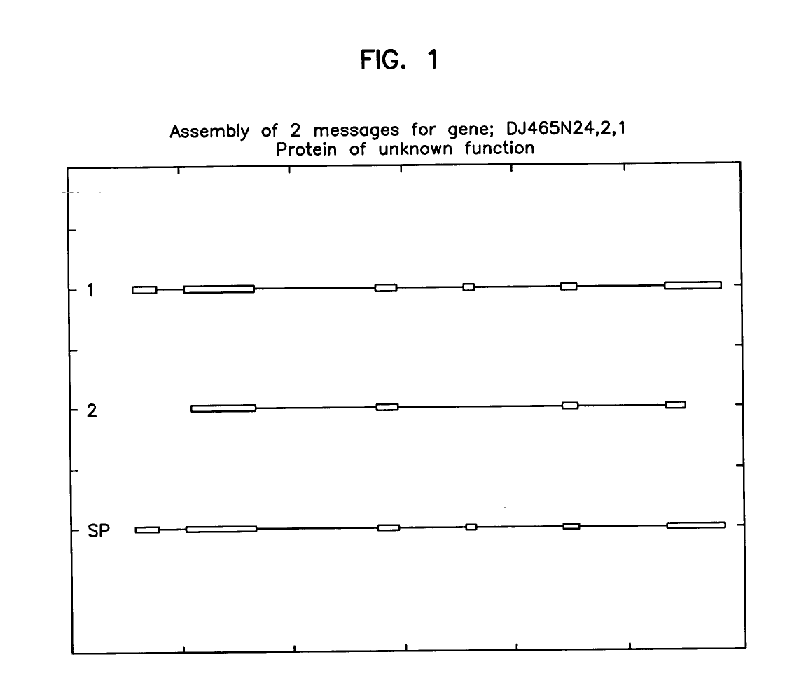

FIGS. 1 through 3 schematically illustrate transcripts and standard polynucleotides for three genes. FIG. 1 schematically illustrates a gene encoding two splice variant mRNAs, labeled 1 and 2. This figure also schematically illustrates a standard polynucleotide (SP) including all of the known bases of every known exon for this gene. In this case, mRNA 1 has the same sequence as the standard polynucleotide.

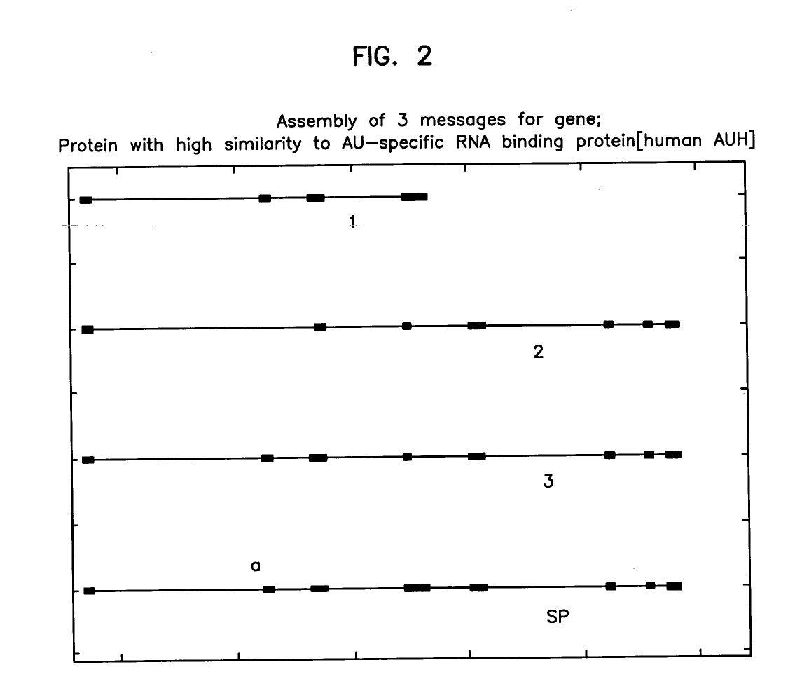

FIG. 2 schematically illustrates a complete exon transcript for a gene encoding three splice variant mRNAs. Each of the illustrated transcripts lacks at least a portion of at least one exon. For example, transcript 1 lacks at least several exons illustrated at the right end of the sequence. Transcript 2 lacks at least a portion of the exon at the right end of transcript 1 and the exon labeled “a” in the standard polynucleotide (SP). Transcript 3 lacks at least a portion of the exon at the right end of transcript 1. That is, the longest encoded transcript, transcript 3, lacks at least a portion of at least one exon, the exon at the right end of transcript 1. The standard polynucleotide (SP) illustrated in FIG. 2 is a complete exon transcript.

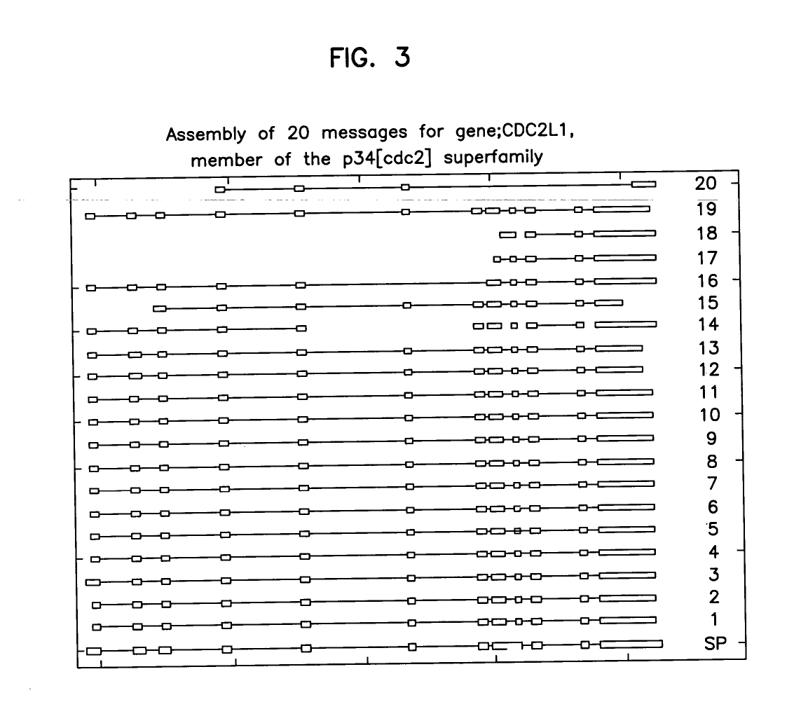

FIG. 3 schematically illustrates a complete exon transcript for a gene encoding 20 splice variant mRNAs. Each of the illustrated transcripts lacks at least a portion of at least one exon. For example, transcripts 1-17 and 18-20 lack at least a portion of the exon illustrated at the left end of transcript 18. Transcript 18 lacks several exons to the left of its terminus. Transcripts 1-13 and 19 appear to be of similar length and each includes at least portions of all of the same exons. However, each of transcripts 1-13 and 19 lack at least a portion of the exon illustrated at the left end of transcript 18. That is, the longest encoded transcript (which is one of transcripts 1-13 or 19) lacks at least a portion of at least one exon, the exon illustrated at the left end of transcript 18. The standard polynucleotide (SP) illustrated in FIG. 3 is a complete exon transcript.

Although not limiting to the present method, it is believed that, a method employing a complete exon transcript can provide one or more of several advantages compared to methods employing conventional standards. These advantages can include: In an embodiment, a single standard polynucleotide according to this embodiment can bind to and detect all known probes for all known splice variants of a gene or message. Thus, a mixture of standard polynucleotides according to this embodiment and including standards corresponding to each gene represented on an array can provide a detectable signal suitable for comparing arrays. Each standard can be present in a mixture in a known quantity, which can allow quantification of the amount of bound standard based on its signal strength. Such a standard can also be employed for quantitating unknown polynucleotide in a sample. The relative amounts of each standard in such a mixture can be selected to provide a measurable signal for each gene of interest. Finally, the identity or quantity of standards present in each mixture can be selected to provide a detectable signal for only genes of interest.

In an embodiment, the present method includes a method of detecting binding to a nucleic acid array. The present method can include contacting the nucleic acid array with a standard polynucleotide for the gene. The standard polynucleotide includes at least one complete exon transcript for a gene. The method can include contacting with a predetermined concentration or amount of the standard polynucleotide. The method can include binding of the standard to a nucleic acid in a feature at a location on the array. Monitoring for binding can employ an apparatus suitable for detecting binding that occurs. Should binding occur, the method can include detecting binding of the standard polynucleotide to the array. This embodiment of the method can also include providing a nucleic acid array. The nucleic acid array can include a plurality of features at locations on a substrate. The present standard can be employed in either multi-channel or single channel microarray-based studies.

The nucleic acid array employed in the present method can include a plurality of locations. A plurality of these locations can include a feature that can bind a polynucleotide. For example, a feature can bind a gene, a transcript of the gene, a polynucleotide including a sequence from the gene, or a complement thereof. The feature can include an oligonucleotide or polynucleotide.

In an embodiment, the present method can include contacting such an array with a plurality of standard polynucleotides. The method can include contacting with a predetermined concentration or amount of at least one of the standard polynucleotides. The method can include contacting with a predetermined concentration or amount of each of the plurality of standard polynucleotides. The predetermined concentration or amount can be the same for each of the standard polynucleotides or it can be different.

The plurality of standard polynucleotides can include standard polynucleotides for a plurality of the features on the array. The plurality of standard polynucleotides includes at least one complete exon transcript for at least one feature on the array. In an embodiment, the plurality of standard polynucleotides includes one standard polynucleotide for each unique feature on the array.

The plurality of standard polynucleotides can include a standard polynucleotide for a plurality of genes of a cell, tissue, or organism. The plurality of standard polynucleotides can include a standard polynucleotide for each gene of a cell, tissue, or organism. The plurality of standard polynucleotides can include a standard polynucleotide for each of a subset of the genes of a cell, tissue, or organism. The subset of genes can include genes of interest, for example, with respect to development, disease, or disorder of the cell, tissue, or organism. At least one, or all, of the standard polynucleotides can include a detectable label, for example, a first detectable label.

The present method can include contacting the nucleic acid array with a sample suspected of containing a polynucleotide that can bind to a feature on the array. The sample polynucleotide can be or include, for example, a gene, a transcript of the gene, a polynucleotide including a sequence from the gene, or a complement thereof. The method can include binding of the sample polynucleotide to a nucleic acid in a feature at a location on the array. Monitoring for binding can employ an apparatus suitable for detecting binding that occurs. Should binding occur, the method can include detecting binding of the sample polynucleotide to the array.

In an embodiment, the present method can include contacting the nucleic acid array with a sample including a plurality of sample polynucleotides. At least one, or all, of the sample polynucleotides can be capable of or suspected of being capable of binding to one or more features on the array. The plurality of sample polynucleotides can each be from the same cell, tissue, or organism. The plurality of sample polynucleotides can include polynucleotides of interest, for example, with respect to development, disease, or disorder of the cell, tissue, or organism. The plurality of sample polynucleotides can be or include a complete set of or a subset of transcripts of genes expressed by a cell, tissue, or organism. At least one, or all, of the sample polynucleotides can include a detectable label, for example, a second detectable label.

In an embodiment, the present method can include contacting each of a plurality of corresponding arrays with the same standard polynucleotide or with the same combination of standard polynucleotides and detecting binding of the standard to the arrays. The method can then include comparing the amount of binding detected at each corresponding location or feature on two or more of the arrays. Standardizing the arrays can be based on this comparison.

In an embodiment, the present method can include detecting a first detectable signal (e.g., color) from the standard polynucleotide and a second detectable signal from a sample polynucleotide. The method can include comparing the strength of the first and second detectable signals. Quantitating the sample polynucleotide can be based on this comparison.

Contacting can include any of a variety of known methods for contacting an array with a reagent, sample, or composition. For example, the method can include placing the array in a container and submersing the array in or covering the array with the reagent, sample, or composition. The method can include placing the array in a container and pouring, pipetting, or otherwise dispensing the reagent, sample, or composition onto features on the array. Alternatively, the method can include dispensing the reagent, sample, or composition onto features of the array, with the array being in or on any suitable rack, surface, or the like.

Detecting can include any of a variety of known methods for detecting a detectable signal from a feature or location of an array. Any of a variety of known, commercially available apparatus designed for detecting signals of or from an array can be employed in the present method. Such an apparatus or method can detect one or more of the detectable labels described hereinbelow. For example, known and commercially available apparatus can detect calorimetric, fluorescent, or like detectable signals of an array. Surface plasmon resonance can be employed to detect binding of a standard polynucleotide to the array. The methods and systems for detecting a signal from a feature or location of an array can be employed for monitoring or scanning the array for any binding that occurs and results in a detectable signal. Monitoring or detecting can include viewing (e.g., visual inspection) of the array by a person.

Methods Employing a Differential Exon Transcript

In an embodiment, the standard polynucleotide can include contiguous nucleotides of a complete exon transcript but less than the entire complete exon transcript. The complete exon transcript includes contiguous sequences surrounding each splice made to produce the multiple transcripts. The smaller standard polynucleotide can include about 50 or more contiguous nucleotides surrounding a splice. The 50 or more contiguous nucleotides are contiguous nucleotides found in a first transcript but not found in a second transcript. Such a standard sequence can be employed to provide a detectable signal for the first transcript but not for the second transcript. In an embodiment, the first transcript can be representative of a first subset of transcripts. Then the standard sequence can provide a detectable signal for the presence of the first subset of transcripts. This embodiment of the standard polynucleotide is referred to herein as a “differential exon transcript” of the gene. The differential exon transcript can be a polynucleotide such as RNA or DNA and can be a plus sense strand or a minus sense strand.

Although not limiting to the present method, it is believed that, a method employing a differential exon transcript can provide one or more of several advantages compared to methods employing conventional standards. These advantages can include one or more of the advantages described above for complete exon transcripts. These advantages can also include: The method can employ a plurality of differential exon transcripts for a particular gene or transcript. In this case, the method can distinguish and/or represent the relative amounts of different splice variants of the gene.

In this embodiment, the present method can include any of the procedures described above. This embodiment can also include contacting the nucleic acid array with a standard polynucleotide for a gene, the standard polynucleotide including at least one differential exon transcript for the gene. The method can include binding of the differential exon transcript to a nucleic acid in a feature at a location on the array. Monitoring for binding can employ an apparatus suitable for detecting binding that occurs. Should binding occur, the method can include detecting binding of the differential exon transcript to the array.

In an embodiment, the present method can include contacting such an array with a plurality of standard polynucleotides. The plurality of standard polynucleotides can include standard polynucleotides for a plurality of the features on the array. The plurality of standard polynucleotides includes at least one differential exon transcript for at least one feature on the array. The plurality of standard polynucleotides can include a standard polynucleotide for each gene of a cell, tissue, or organism. The plurality of standard polynucleotides can include a standard polynucleotide for each of a subset of the genes of a cell, tissue, or organism. The subset of genes can include genes of interest, for example, with respect to development, disease, or disorder of the cell, tissue, or organism. At least one, or all, of the standard polynucleotides can include a detectable label, for example, a first detectable label.

Complete or Differential Exon Transcripts and Compositions

An embodiment includes a polynucleotide such as a complete exon transcript. Another embodiment includes a composition including at least one complete exon transcript. A composition including a complete exon transcript can also include one or more additional standard polynucleotides, such as a differential exon transcript; the mRNA, cDNA, or a complement thereof for a gene that is expressed as only a single transcript; the mRNA, cDNA, or complement thereof that includes all of the known bases of all of the known exons for a gene with such a transcript; or a mixture thereof. The composition can also include a carrier for the one or more polynucleotides, such as an aqueous buffer, a solvent, or the like.

The present composition can include standard polynucleotides for a plurality of genes of a cell, tissue, or organism. In an embodiment, the present composition can include standard polynucleotides for each gene of a cell, tissue, or organism. Alternatively, the present composition can include standard polynucleotides for a subset of the genes of a cell, tissue, or organism. The subset of genes can include genes of interest, for example, with respect to development, disease, or disorder of the cell, tissue, or organism. The present composition can include standard polynucleotides for a plurality of the features on an array.

The present standard polynucleotides or compositions can be provided in any variety of common formats. The present nucleotide or composition can be provided in a container, for example, as a solid (e.g., a lyophilized solid) or a liquid. In an embodiment, each of a plurality of standard polynucleotides is provided in its own container (e.g., vial, tube, or well). The present standard polynucleotides or compositions can be provided with materials for creating a nucleic acid array or with a complete nucleic acid array. In fact, the present polynucleotide or composition can be provided bound to one or more features of a nucleic acid array.

Differential Exon Transcript Compositions

An embodiment includes a composition including at least one differential exon transcript. A composition including a differential exon transcript can also include one or more additional standard polynucleotides, such as a complete exon transcript; the mRNA, cDNA, or a complement thereof for a gene that is expressed as only a single transcript; the mRNA, cDNA, or complement thereof that includes all of the known bases of all of the known exons for a gene with such a transcript; or a mixture thereof. This embodiment of the composition can also include a carrier for the one or more polynucleotides, such as an aqueous buffer, a solvent, or the like. The present differential exon transcript can include a detectable label, for example, a first detectable label.

This embodiment of the present composition can include differential exon transcripts or standard polynucleotides for a plurality of genes of a cell, tissue, or organism. In an embodiment, the present composition can include differential exon transcripts or standard polynucleotides for each gene of a cell, tissue, or organism. Alternatively, the present composition can include differential exon transcripts or standard polynucleotides for a subset of the genes of a cell, tissue, or organism. The subset of genes can include genes of interest, for example, with respect to development, disease, or disorder of the cell, tissue, or organism. The present composition can include differential exon transcripts or standard polynucleotides for a plurality of the features on an array.

In an embodiment, the differential exon transcript is at least about 30 nucleotides in length, at least about 60 nucleotides in length, at least about 90 nucleotides in length, at least about 120 nucleotides in length, at least about 150 nucleotides in length, at least about 180 nucleotides in length, at least about 210 nucleotides in length, at least about 240 nucleotides in length, at least about 270 nucleotides in length, at least about 300 nucleotides in length, at least about 450 nucleotides in length, at least about 600 nucleotides in length, at least about 900 nucleotides in length, or more, and specifically excludes naturally occurring transcripts.

Additional Standard Polynucleotides

In one aspect, the standard nucleic acid molecule includes a nucleotide sequence having at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, or at least about 99% sequence identity to the complete exon transcript, the differential exon transcript, or the complement thereof.

In an embodiment, the present standard polynucleotide can be described as hybridizing to the present complete exon transcript, the present differential exon transcript, or the complement thereof, for example, under stringent hybridization and wash conditions.

Detectable Labels

The present standard polynucleotide, complete exon transcript, or differential exon transcript can include a detectable label, for example, a first detectable label. Sample polynucleotides can include a detectable label, for example, a second detectable label. Suitable labels include radioactive labels and non-radioactive labels, directly detectable and indirectly detectable labels, and the like. Directly detectable labels provide a directly detectable signal without interaction with one or more additional chemical agents. Suitable of directly detectable labels include colorimetric labels, fluorescent labels, and the like. Indirectly detectable labels interact with one or more additional members to provide a detectable signal. Suitable indirect labels include a ligand for a labeled antibody and the like.

Suitable fluorescent labels include any of the variety of fluorescent labels disclosed in United States Patent Application Publication No. 20010009762, the disclosure of which is incorporated herein by reference. Specific suitable fluorescent labels include: xanthene dyes, e.g., fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g., Cy3, Cy5 and Cy7 dyes; coumarins, e.g., umbelliferone; benzimide dyes, e.g., Hoechst 33258; phenanthridine dyes, e.g., Texas Red; ethidium dyes; acridine dyes; carbazole dyes; phenoxazine dyes; porphyrin dyes; polymethine dyes, e.g., cyanine dyes such as Cy3, Cy5, etc; BODIPY dyes and quinoline dyes.

Methods of Making Complete or Differential Exon Transcripts and Compositions

The present standard polynucleotides can be produced by known methods. For example, the standard polynucleotides can be made by known methods for chemical synthesis. Chemical synthesis can employ commercial synthesizers. Chemical synthesis can produce thousands or more different sequences in multi-well plates. The method can include synthesizing segments of the polynucleotide and ligating the segments to form the polynucleotide. Chemical synthesis can be employed to make a complete exon transcript or a differential exon transcript.

The standard polynucleotides can be made by known recombinant methods. For example, conventional cloning and subcloning techniques and PCR can be employed to produce the standard polynucleotides. In an embodiment, exons can be isolated from biological samples by PCR and ligated together to produce a complete exon transcript or a differential exon transcript.

Recombinant Methods

DNA encoding exons of interest can be obtained from a suitable cDNA or genomic library. The libraries can be screened with probes designed to identify the exon of interest according to standard procedures. See, e.g., Sambrook et al., Molecular Cloning: A Laboratory Manual (New York: Cold Spring Harbor Laboratory Press, 1989). The probe can be labeled according to known methods so that it can be detected upon hybridization to DNA in the library. The exons can be isolated and ligated using known methods.

Host cells can be modified to produce the standard polynucleotide by transfecting or transforming with a cloning vector according to known techniques. The transfecting or transformed cells can be cultured in conventional nutrient media, which can be modified inducing promoters, selecting transformants, or amplifying the genes encoding the desired sequences. Suitable culture conditions (e.g., media, temperature, pH) can be chosen without undue experimentation. Suitable techniques for cell culture are known. See, e.g., Mammalian Cell Biotechnology: a Practical Approach, M. Butler, ed. (IRL Press, 1991) and Sambrook et al., supra.

Transfection can employ known methods appropriate for each host cell type. Suitable methods include CaCl₂, CaPO₄, liposome-mediated, or electroporation. Other known methods for introducing DNA into cells can be used. Suitable host cells for cloning or expressing DNA in vectors include prokaryote (e.g., E. coli), filamentous fungi or yeast, or higher eukaryote (e.g., insect or mammalian) cells. Such host cells are well-known and are publicly and even commercially available.

The DNA encoding the standard polynucleotide can be inserted into a replicable vector for amplification of the DNA. Suitable vectors are well-known and are publicly and even commercially available. The vector can, for example, be in the form of a plasmid, cosmid, virus particle, or phage. The appropriate nucleic acid sequence may be inserted into the vector by any of a variety of known procedures. The vector can also include, for example, a signal sequence, an origin of replication, a marker gene, an enhancer element, a promoter, and/or a transcription termination sequence. Such elements and sequences are known. The vector can be constructed employing well-known techniques. Gene amplification can be detected or measured in a host cell or other system by any of a variety of known methods (e.g., Southern blotting, Northern blotting, dot blotting, or in situ hybridization).

Arrays

Arrays on a substrate can be designed for testing against any type of sample, whether a trial sample, reference sample, a combination of them, or a known mixture of polynucleotides (in which latter case the arrays may be composed of features carrying unknown sequences to be evaluated). Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 50 cm², 20 cm², or even less than 10 cm², or less than 1 cm². For example, features may have widths (that is, diameter, for a round spot) in the range from a 10 μm to 1.0 cm. In other embodiments each feature may have a width in the range of 1.0 μm to 1.0 mm, of 5.0 μm to 500 μm, or of 10 μm to 200 μm. Non-round features may have area ranges equivalent to that of circular features with the foregoing width (diameter) ranges. Feature sizes can be adjusted as desired, for example by using one or a desired number of pulses from a pulse jet to provide the desired final spot size.

At least some, or all, of the features are of different compositions (for example, when any repeats of each feature composition are excluded the remaining features may account for at least 5%, 10%, or 20% of the total number of features), each feature typically being of a homogeneous composition within the feature. Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Array features will generally be arranged in a regular pattern (for example, rows and columns). However other arrangements of the features can be used where the user has, or is provided with, some means (for example, through an array identifier on the array substrate) of being able to ascertain at least information on the array layout (for example, any one or more of feature composition, location, size, performance characteristics in terms of significance in variations of binding patterns with different samples, or the like). Each array feature is generally of a homogeneous composition.

Each array may cover an area of less than 100 cm², or even less than 50 cm², 10 cm², or 1 cm². In many embodiments, the substrate carrying the one or more arrays will be shaped generally as a rectangular solid (although other shapes are possible), having a length of more than 4 mm and less than 1 m, for example, more than 4 mm and less than 600 mm, less than 400 mm, or less than 100 mm; a width of more than 4 mm and less than 1 m, for example, less than 500 mm, less than 400 mm, less than 100 mm, or 50 mm; and a thickness of more than 0.01 mm and less than 5.0 mm, for example, more than 0.1 mm and less than 2 mm, or more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse jets of either polynucleotide precursor units (such as monomers) in the case of in situ fabrication, or the previously obtained polynucleotide. Such methods are described in detail in, for example, U.S. Pat. Nos. 6,656,740; 6,458,583; 6,323,043; 6,372,483; 6,242,266; 6,232,072; 6,180,351; 6,171,797; or 6,323,043; or in U.S. patent application Ser. No. 09/302,898 filed Apr. 30, 1999 by Caren et al., and the references cited therein. These references are incorporated herein by reference. Other drop deposition methods can also be used for fabrication. Also, instead of drop deposition methods, known photolithographic array fabrication methods may be used. Interfeature areas need not be present particularly when the arrays are made by photolithographic methods as described in those patents.

Methods Employing Arrays

Following receipt by a user of an array made by an apparatus or method of the present invention, it will typically be exposed to a sample (for example, a fluorescently labeled polynucleotide or protein containing sample) in any well known manner and the array is then read. Reading of the array may be accomplished by illuminating the array and reading the location and intensity of resulting fluorescence at multiple regions on each feature of the array. For example, a scanner may be used for this purpose which is similar to the AGILENT MICROARRAY SCANNER manufactured by Agilent Technologies, Palo Alto, Calif. Other suitable apparatus and methods are described in U.S. patent applications: Ser. No. 10/087447 “Reading Dry Chemical Arrays Through The Substrate” by Corson et al.; and in U.S. Pat. Nos. 6,592,036; 6,583,424; 6,486,457; 6,406,849; 6,371,370; 6,355,921; 6,320,196; 6,251,685; and 6,222,664. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. Nos. 6,251,685, or 6,221,583 and elsewhere). Data from read arrays may be processed in any know manner, such as described in U.S. Pat. No. 6,591,196, U.S. patent application Ser. No. 09/659,415 filed Sep. 11, 2000 for “Method And System For Extracting Data From Surface Array Deposited Features”, and many commercially available array feature extraction software packages. A result obtained from the reading followed by a method of the present invention may be used in that form or may be further processed to generate a result such as that obtained by forming conclusions based on the pattern read from the array (such as whether or not a particular target sequence may have been present in the sample, or whether or not a pattern indicates a particular condition of an organism from which the sample came). A result of the reading (whether further processed or not) may be forwarded (such as by communication) to a remote location if desired, and received there for further use (such as further processing).

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

What is claimed is:

1. A method of detecting binding to a nucleic acid array, the method comprising:
providing a nucleic acid array comprising a plurality of features at locations on a substrate;
contacting the nucleic acid array with a standard polynucleotide for a gene; the standard polynucleotide comprising a complete exon transcript of the gene; and
monitoring for detectable binding of the standard polynucleotide to the array.

2. The method of claim 1, further comprising:
contacting the nucleic acid array with a sample suspected of containing the gene, transcript of the gene, or polynucleotide including a sequence from the gene.

3. The method of claim 1, comprising contacting the array with a plurality of standard nucleotides; at least one of the standard polynucleotides comprising a complete exon transcript.

4. The method of claim 3, further comprising:
contacting the nucleic acid array with a sample suspected of containing at least one gene, transcript of the gene, or polynucleotide including a sequence from the gene.

5. The method of claim 4, wherein the sample comprises transcripts of genes expressed by an organism expressing splice variants of a first gene.

6. The method of claim 5, comprising:
contacting the nucleic acid array with a first sample suspected of containing a first set of splice variants of the first gene; and
contacting the nucleic acid array with a second sample suspected of containing a second set of splice variants of the first gene.

7. The method of claim 5, wherein the plurality of standard polynucleotides comprise a standard polynucleotide for each gene of the organism.

8. The method of claim 4, comprising wherein the sample comprises transcripts of genes expressed by a cell, a cell type, an individual, a species, a genus, or an order.

9. The method of claim 8, wherein the plurality of standard polynucleotides comprise a standard polynucleotide for one or more genes expressed by the cell, the cell type, the individual, the species, the genus, or the order.

10. The method of claim 9, wherein the plurality of standard polynucleotides comprise standard polynucleotides for a particular subset of exons or splice variants expressed by the cell, the cell type, the individual, the species, the genus, or the order.

11. The method of claim 4, comprising wherein the sample comprises transcripts of genes expressed by an organism under a growth condition, an environmental condition, or a developmental stage.

12. The method of claim 11, wherein the plurality of standard polynucleotides comprise a standard polynucleotide for one or more genes expressed by the organism under the growth condition, the environmental condition, or the developmental stage.

13. The method of claim 12, wherein the plurality of standard polynucleotides comprise standard polynucleotides for a particular subset of exons or splice variants expressed by the organism under the growth condition, the environmental condition, or the developmental stage.

14. The method of claim 4, wherein the plurality of standard polynucleotides comprise a standard polynucleotide for each gene of the organism.

15. The method of claim 4, further comprising:
detecting binding to the array by the gene, transcript of the gene, or polynucleotide including a sequence from the gene.

16. The method of claim 4, further comprising labeling each nucleic acid in the sample.

17. The method of claim 9, further comprising detecting binding to the array by at least one labeled nucleic acid from the sample.

18. The method of claim 3, wherein the plurality of standard polynucleotides comprise a label.

19. A method of detecting binding to a nucleic acid array, the method comprising:
providing a nucleic acid array comprising a plurality features at locations on a substrate;
contacting the nucleic acid array with a standard polynucleotide for a gene; the standard polynucleotide comprising a differential exon transcript of the gene; and
monitoring for detectable binding of the standard polynucleotide to the array.

20. The method of claim 19, further comprising:
contacting the nucleic acid array with a sample suspected of containing the gene, transcript of the gene, or polynucleotide including a sequence from the gene.

21. The method of claim 20, further comprising:
detecting binding to the array by the gene, transcript of the gene, or polynucleotide including a sequence from the gene.

22. A method of making a polynucleotide, the method comprising:
determining the sequence of nucleotides making up a complete exon transcript for a gene;
producing a polynucleotide comprising the sequence of nucleotides of the complete exon transcript.

23. The method of claim 22, wherein producing comprises chemically synthesizing the polynucleotide.

24. The method of claim 23, further comprising synthesizing segments of the polynucleotide and ligating the segments to form the polynucleotide.

25. The method of claim 22, wherein producing comprises recombinant production of the polynucleotide.

26. A method of making a polynucleotide, the method comprising:
determining the sequence of nucleotides making up a differential exon transcript for a gene;
producing a polynucleotide comprising the sequence of nucleotides of the differential exon transcript.

27. The method of claim 26, wherein producing comprises chemically synthesizing of the polynucleotide.

28. The method of claim 27, further comprising synthesizing segments of the polynucleotide and ligating the segments to form the polynucleotide.

29. The method of claim 26, wherein producing comprises recombinant production of the polynucleotide.

30. A composition comprising:
a polynucleotide comprising a complete exon transcript for a gene;
a polynucleotide comprising a differential exon transcript for a gene; or
a combination thereof.

31. The composition of claim 30, comprising a plurality of polynucleotides.

32. The composition of claim 31, comprising:
a standard polynucleotide for each gene or an organism;
the standard polynucleotides comprising a complete exon transcript for a gene, a differential exon transcript for a gene, or a combination thereof.

33. The composition of claim 30, further comprising a nucleic acid array.

34. The composition of claim 30, further comprising a buffer composition.

35. A composition comprising:
a nucleic acid array; and
a polynucleotide comprising a complete exon transcript for a gene;
the polynucleotide being bound to the array.

36. A composition comprising:
a nucleic acid array; and
a polynucleotide comprising a differential exon transcript for a gene;
the polynucleotide being bound to the array.

37. A method of making a standard polynucleotide, comprising:
determining the sequence of nucleotides making up a complete exon transcript for a gene;
providing the sequence of the complete exon transcript in a format readable by a machine or by a human.

38. The method of claim 37, comprising determining and providing the sequences of a plurality of complete exon transcripts.

39. The method of claim 37, further comprising:
determining the sequence of nucleotides making up a differential exon transcript for a gene;
providing the sequence of the differential exon transcript in a format readable by a machine or by a human.

40. A method of making a standard polynucleotide, comprising:
determining the sequence of nucleotides making up a differential exon transcript for a gene;
providing the sequence of the differential exon transcript in a format readable by a machine or by a human.

41. A method comprising forwarding to a remote location a result obtained by the method of claim 1.

42. A method comprising forwarding to a remote location a result obtained by the method of claim 19.

43. A method comprising transmitting data representing a result of a reading obtained by the method of claim 1.

44. A method comprising transmitting data representing a result of a reading obtained by the method of claim 19.
